(19) United States
(12) Patent Application Publication
(10) Pub. No.: US 2025/0135060 A1
(43) Pub. Date: May 1, 2025

(54) DEVICE FOR REMOVING VIRUS PARTICLES IN AN AIR FLOW

(71) Applicants: William Thomas Crocca, Rochester, NY (US); Fred Christopher Zusi, Hamden, CT (US); UNIVERSITA' DEGLI STUDI DI PAVIA, Pavia (IT)

(72) Inventors: William Thomas Crocca, Rochester, NY (US); Fred Christopher Zusi, Hamden, CT (US); Hellas Cena, Venegono Superiore (VA) (IT); Hermes Giberti, Bergamo (IT)

(21) Appl. No.: 18/835,196

(22) PCT Filed: Jan. 30, 2023
(86) PCT No.: PCT/IB2023/050790

§ 371 Date: Aug. 1, 2024

(30) Foreign Application Priority Data
Feb. 1, 2022 (IT) 102022000001622

(51) Int. Cl. A61L 9/14; A61L 101/32

(52) U.S. Cl. CPC A61L 9/145; A61L 2101/32; A61L 2209/14

(57) ABSTRACT

CN210373855: The utility model provides an air purifier relates to air purification technical field, and this air purifier includes: the liquid storage tank is used for storing decomposition liquid, the liquid storage tank is connected with the upper end of the filtering structure, and a plurality of first leakage holes are formed in the connection position of the liquid storage tank and the filtering structure; the air purifier comprises an air guide channel, and the filtering structure is positioned in the air guide channel and used for filtering air passing through the air guide channel. The air purifier can comprehensively finish sterilization, deodorization, formaldehyde removal and the like, and the decomposition liquid can adsorb smoke floating dust, haze and suspended particles in the air, and is matched with the filtering structure to be beneficial to comprehensively removing particle pollutants in the air; and compared with the traditional filter element, the filter element has the advantages of low price of filtrate, low material consumption and low use cost.

### TECHNICAL FIELD

The subject-matter disclosed herein relates to a device for removing virus particles in an air flow.

### BACKGROUND ART

The transmission of many viruses, including SARS-CoV-2, occurs mainly through the dispersion of droplet particles and by inhalation of aerosol particles. It is recognized that good hygiene practices and correct use of personal protective equipment (PPE) are, together with social distancing, the major tools currently effective in preventing the spread of this kind of disease. Moreover, specific actions must be considered taking into account environmental contamination as a possible source of virus infection. In this context, environment means surfaces and indoor air. Monitoring the indoor air is of major importance for the protection and prevention of the health of citizens and workers.

The existing N95 masks operate by directing the wearer's inhaled air through a 5-micron mesh that filters out all particles 5 microns and larger. The SARS-CoV-2 virus is only 3 microns in size, therefore multiple virus particles stuck together will not pass through the N95 mesh, but the occasional single virus particle will pass. It is to be noted that only one single virus particle is sufficient to infect a victim. Therefore, it is necessary to develop improved useful tools and devices for managing, preventing and contrasting conditions of increased virus infection risk both in healthcare environment and in indoor areas (for example public transport, offices . . . ).

### SUMMARY

According to an aspect, the subject-matter disclosed herein relates to an innovative device, advantageously used as or integrated in a Personal Protective Equipment (PPE), in particular in a Respiratory (personal) Protective Equipment (RPE), and being wearable by a user, for removing virus particles in an air flow that, rather than attempting to mechanically filter virus particles from the air flow, submerges them in a benign surfactant liquid solution (as opposed to either a toxic and/or corrosive solution) that dissolves the fatty outer “skin” of the virus and therefore causing the exposure and disassembly of its internal proteins, thus destroying the virus altogether. However, the destruction process is not instantaneous and the time that is necessary for the destruction to occur depends on a variety of factors. The innovative device comprises a specialized tubular member and a gas-permeable, liquid-impermeable membrane, in particular water-impermeable, for example Polytetrafluoroethylene (PTFE), in order to contain the surfactant liquid and trap the virus in the liquid for the necessary time, leaving the purified air to pass. Advantageously, the power to drive the incoming airflow through the innovative device comes from the user as he/she inhales.

### BRIEF DESCRIPTION OF THE DRAWINGS

A more complete appreciation of the disclosed embodiments of the invention and many of the attendant advantages thereof will be readily obtained as the same becomes better understood by reference to the following detailed description when considered in connection with the accompanying drawings, wherein:

FIG. 1 shows a simplified view of an embodiment of a tubular member according to the present invention,

FIG. 2 shows the tubular member of FIG. 1 surrounded by an envelope,

FIG. 3 shows a cross-sectional view of a first embodiment of a tubular member surrounded by an envelope,

FIG. 4 shows a cross-sectional view of a second embodiment of a tubular member surrounded by an envelope,

FIG. 5 shows a cross-sectional view of a third embodiment of a tubular member surrounded by an envelope, and

FIG. 6 shows a simplified view of an embodiment of an innovative device for removing virus particles in an air flow according to the present invention including a casing, a filter and a one-way valve during an inspiration phase.

### DETAILED DESCRIPTION OF EMBODIMENTS

According to an aspect, the subject-matter disclosed herein relates to a device, advantageously used in a Personal Protective Equipment (PPE), in particular in a Respiratory (personal) Protective Equipment (RPE), and being wearable by a user, for removing virus particles in an air flow by passing the air flow through a virucidal liquid, in particular a surfactant, more in particular a benzalkonium chloride solution; advantageously, the virucidal liquid is a 5% by weight aqueous solution of benzalkonium chloride. The device comprises a tubular member in which the air enters through an inlet at an end portion of the tubular member, flows in the tubular member, escapes through a plurality of openings at an outer lateral surface of the tubular member and diffuses through the virucidal liquid which is contained in an envelope that surrounds totally or partially the tubular member. The envelope comprises a gas-permeable, liquid-impermeable membrane, in particular water-impermeable, through which the air flow can exit after virus removal. It is to be noted that it takes some time for the air to diffuse inside the envelope and to exit from its membrane so that the virucidal liquid has time for acting on the air containing any virus.

Reference now will be made in detail to embodiments of the disclosure, examples of which are illustrated in the drawings. Each example is provided by way of explanation of the disclosure, not limitation of the disclosure. In fact, it will be apparent to those skilled in the art that various modifications and variations can be made in the present disclosure without departing from the scope or spirit of the disclosure. In the following description, similar reference numerals are used for the illustration of figures of the embodiments to indicate elements performing the same or similar functions.

The subject-matter disclosed herein relates to a device for removing virus particles in an air flow. The device is generally indicated with reference numeral 100. The device 100 comprises a tubular member 10 and an envelope 20 which surrounds totally or partially the tubular member 10 and which contains a virucidal liquid. The envelope 20 comprises a gas-permeable and liquid-impermeable membrane, in particular water-impermeable, so that the air enters the device 100 through the tubular member 10, diffuses into the virucidal liquid which removes virus particles and, after virus removal, the air flow exits from the device 100 through the gas-permeable and liquid-impermeable membrane. It is to be noted that the feature “water-impermeable” of the membrane it is intended to include not only water but also any contaminants, such as solids and bacteria or viruses, present in the water.

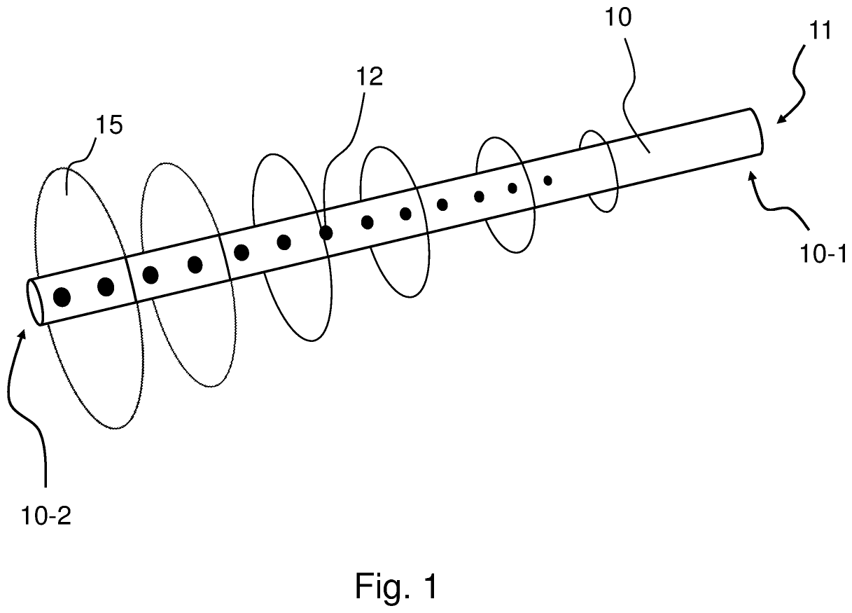

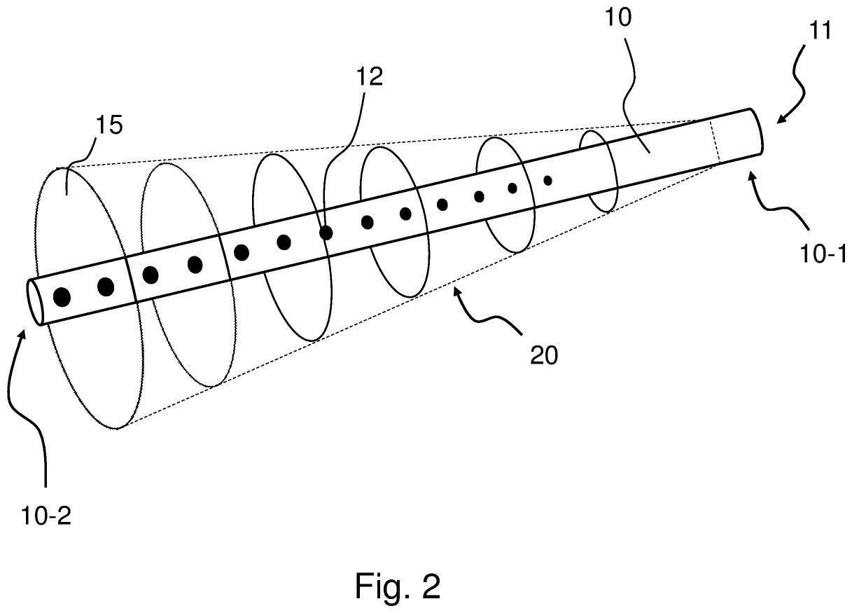

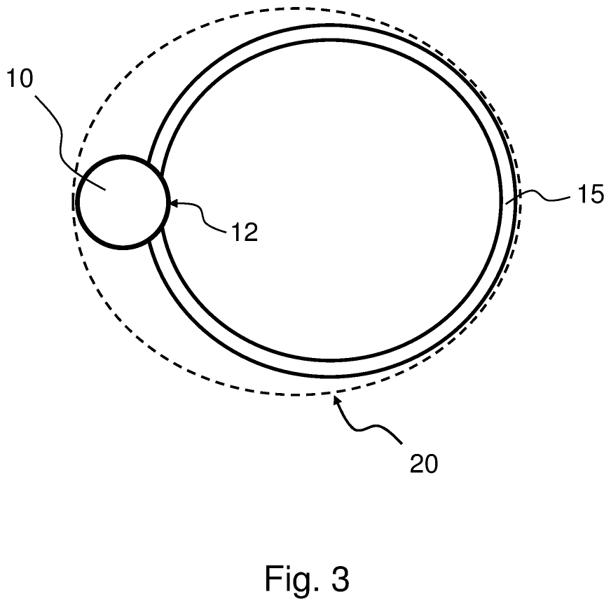

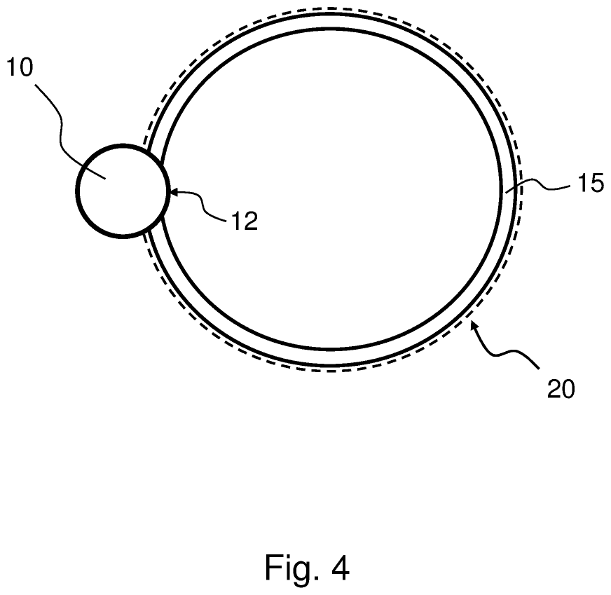

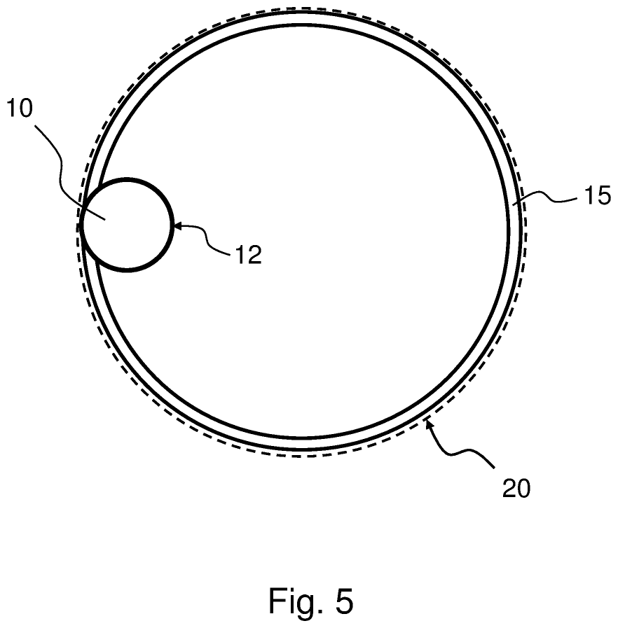

In FIG. 1 is schematically shown a preferred embodiment of the tubular member 10, in FIG. 2 is schematically shown the embodiment of the tubular member 10 of FIG. 1 surrounded by the envelope 20 and in FIG. 3, FIG. 4 and FIG. 5 are shown cross-sectional views of three different embodiments of a tubular member surrounded by an envelope. The tubular member 10 has a first end 10-1 and a second end 10-2; an inlet 11 of the tubular member 10, through which the air can enter in the device 100, is located at the first end 10-1; in particular, the air enters via the inlet 11 and flows through the device 100 during an inspiration phase. As it will be apparent from the following, the tubular member 10 further comprises means, preferably located at the first end 10-1, for preventing the exit of virucidal liquid from the inlet 11 during an exhalation phase, for example a one-way valve. Advantageously, the second end 10-2 is closed. The tubular member 10 has a plurality of outlets 12 at an outer lateral surface, in particular on the outer lateral surface of the tubular member 10 between the first end 10-1 and the second end 10-2. For example, during an inspiration phase, the air enters the device 100 through inlet 11, flows in the tubular member 10 and escapes through the outlets 12.

Advantageously, the outlets 12 are holes which have an increasing size, in particular an increasing diameter, depending on a distance from the inlet 11. Preferably, the outlets 12 which are farther from the first end 10-1 of the tubular member 10 have larger size (e.g. diameter) than the outlets 12 which are closer to the first end 10-1 of the tubular member 10. Advantageously, increasing the size (e.g. diameter) of the outlets 12 as moving away from the inlet 11 may compensate the increasing of “back-pressure”, which is a typical phenomenon that occurs in pipes through which a fluid flows, through the tubular member 10 from the first end 10-1, where the air enters, to the second end 10-2. In particular, increasing the size (e.g. diameter) of the outlets 12 as moving away from the inlet 11 may reduce and/or compensate the resistance to the inlet air due to back-pressure.

Advantageously, the tubular member 10 comprises further a plurality of ribs 15 configured to support the envelope 20. In particular, the ribs 15 are mechanically coupled to the outer lateral surface of the tubular member 10. In particular, the ribs 15 are attached to the outer lateral surface of the tubular member 10 so that the tubular member 10 may be considered as a spine for the ribs 15. For example, in FIG. 1 and FIG. 2 is shown an embodiment of tubular member 10 with six ribs 15. Advantageously, with non-limiting reference to FIG. 3, FIG. 4 and FIG. 5, the ribs 15 are rings, in particular circular rings; alternatively, the ribs may be disks, in particular circular disks. According to a first embodiment shown in FIG. 3 and a second embodiment shown in FIG. 4, the ribs 15 are attached to the outer lateral surface of the tubular member 10 in a region near the outlets 12 and the tubular member 10 is at least partially external to the ribs 15. According to a third embodiment shown in FIG. 5, the ribs 15 are attached to the outer lateral surface of the tubular member 10 in a region far from the outlets 12 and the tubular member 10 is substantially internal to the ribs 15.

Advantageously, the ribs 15 have an increasing size, in particular an increasing diameter, depending on a distance from the inlet 11. Preferably, the ribs 15 which are farther from the first end of the tubular member 10 have larger size (e.g. diameter) than the ribs 15 which are closer to the first end of the tubular member 10.

As mentioned above, the device 100 comprises also the envelope 20 which surrounds the tubular member 10 and which contains a virucidal liquid. Advantageously, the envelope 20 is in the form of a sack having a mouth sealed to the first end portion 10-1 of the tubular member 10. It is to be noted that the inlet 11 of the tubular member 10 is external to the envelope 20, so that the envelope 20 is fluidly coupled to the surrounding ambient atmosphere through the tubular member 10; in other words, for example during an inspiration phase, the air from the surrounding ambient atmosphere enters into the tubular member 20, bubbles out from the outlets 12 and flows into the envelope 20. With non-limiting reference to FIG. 2, since advantageously the envelope 20 is made of a pliable material, in particular a gauze-like material, the envelope 20 is supported by the ribs 15; in other words, the ribs 15 provide a structure to the envelope 20, i.e. cause it to maintain a specific shape. Considering FIG. 3 and FIG. 5, the envelope 20 may surround totally both the tubular member 10 and the ribs 15; alternatively, considering for example FIG. 4, the envelope 20 may surround totally only the ribs 15 and being sealed to the tubular member 10 in a region near the outlets 12, such that the outlets 12 are internal to the envelope 20 (in other words, the outlets 12 are totally surrounded by the envelope 20).

For example, the envelope 20 may have at least a portion made of a gas-permeable and liquid-impermeable membrane, preferably made in Polytetrafluoroethylene. Advantageously, the gas-permeable and liquid-impermeable membrane is positioned so to face at least the outlets 12 (see e.g. FIG. 3, FIG. 4 and FIG. 5). More advantageously, the whole envelope 20 is made of gas-permeable and liquid-impermeable membrane.

For example, with non-limiting reference to FIG. 2, the ribs 15 allow the envelope 20 to be shaped into a cone-like or truncated-cone-like or trumpet-like envelope; advantageously, the shape of the envelope 20 shown in FIG. 2 maximizes the surface area of the gas-permeable and liquid-impermeable membrane which can be passed through by the air flow.

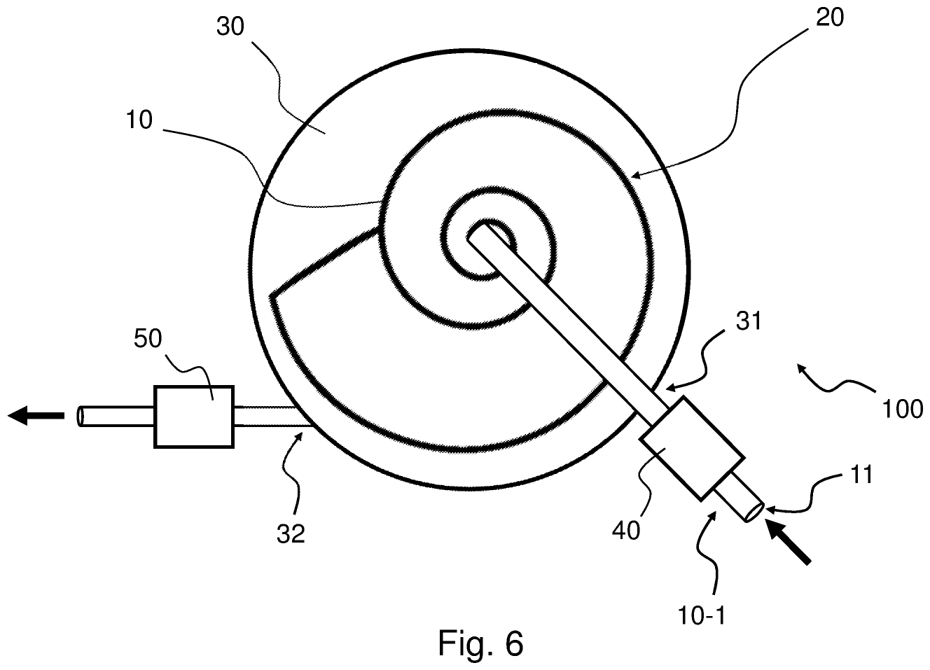

Preferably, the tubular member 10 is made of a flexible material. Advantageously, the tubular member 10 is rolled up so to form in particular a solenoid or a spiral, in particular a logarithmic spiral, such as a Nautilus shape (see for example the simplified view shown in FIG. 6). According to a preferred embodiment shown for example in FIG. 2, the tubular member 10 is surrounded by the envelope 20; it is to be noted that also the envelope 20 may be rolled up, preferably together with the tubular member 10. Advantageously, the shape of the tubular member 10 surrounded by the envelope 20 rolled up together, shown for example in FIG. 6, minimize the volume occupied by the device 100. It is to be noted that the form of solenoid or spiral of the tubular member 10 may be particularly advantageous when the device 100 is integrated in a personal protective equipment (PPE), for example in a face mask. As it will be better described in the following, the device 100 in use as or integrated in a PPE will have a “head space” above the virucidal liquid, in particular inside the envelope 20, and another “head space” outside the envelope 20 and before an outlet port to enable useful air flow. Inhalation by the wearer will create a partial vacuum in both spaces, drawing air through the virucidal liquid. The gas-permeable and liquid-impermeable membrane of the envelope 20 will prevent any liquid from escaping while allowing the air to flow.

It is also to be noted that if the device 100 is implemented in a PPE, the inhalation made by the person wearing the device 100 allows the air entering the device 100 from the inlet 11 and exiting from the gas-permeable and liquid-impermeable membrane. Advantageously, with non-limiting reference to FIG. 6, when a person which is wearing the device 100 embodied in a PPE breathes in, it will create a partial vacuum which draws air through the virucidal liquid. However, the device 100 may also be scaled up for use in buildings, transport, etc.; in those cases, a fan may be added in order to move sufficient air flow.

As mentioned above, the envelope 20 contains a virucidal liquid. Preferably. the virucidal liquid is a surfactant solution, in particular a benzalkonium chloride solution; advantageously, the virucidal liquid is a 5% (weight percent) aqueous solution of benzalkonium chloride (i.e. a 5% by weight benzalkonium chloride solution in which solvent is water).

Advantageously, the device 100 comprises also a casing 30, preferably a rigid casing, which houses the envelope 20 and at least partially the tubular member 10, preferably both in the form of a solenoid or a spiral, in particular a logarithmic spiral (see FIG. 6); in particular, the tubular member 10 is housed into the casing 30 substantially totally, except for the inlet 11. Advantageously, inside the casing there may be one or more devices (not shown in any of the annexed figures) to hold in position the assembly of the tubular member and the envelope. The casing 30 has an inlet port 31 to receive the air flow before virus removal and an outlet port 32 to let the air flow out after virus removal: the inlet port 31 is fluidly coupled to the inlet 11 and the outlet port 32 is fluidly coupled to the gas-permeable and liquid-impermeable membrane of the envelope 20. In particular, the inlet port 31 and the outlet port 32 may be openings of the casing 30 that may accommodate ducts. It is to be noted that the ducts that pass through the inlet port 31 and the outlet port 32 may be considered part for example of the casing 30 or part of the tubular member 10.

For example, with non-limiting reference to FIG. 6, the first end 10-1 of the tubular member 10 may extend outside the casing 30, passing through the inlet port 31 of the casing 30, so that the inlet 11 of the tubular member 10 is located outside the casing 30. Preferably, the first end 10-1 of the tubular member 10 passing through the inlet port 31 is sealed to the inlet port 31, so that no leakage can occur at the inlet port 31 and all the air flow enters in the device 100 through the inlet 11. As schematically shown in FIG. 6, the portion of the tubular member 10 may be axially coupled to the casing 30 so that an axis of the tubular element 10 is substantially coincident with an axis of the casing 30.

For example, with non-limiting reference to in FIG. 6, the air which flows through the gas-permeable and liquid-impermeable membrane after virus removal may exit the casing 30 through the outlet port 32. In particular, the duct that passes through the outlet port 32 is adapted to be fluidly coupled to a mouth and/or a nose of a wearer of the device 100. In particular, an inspiration of a wearer of the device 100 provides the ability to create a vacuum inside the casing 30 so that the device 100 draws air flow from a surrounding ambient atmosphere into the virucidal liquid, the air flow exits through the gas-permeable and liquid-impermeable membrane into the casing 30, and then out the casing 30 through the outlet port 32 as clean air. It is to be noted, as already described above, that the gas-permeable and liquid-impermeable membrane substantially prevents any liquid from escaping while allows the air to flow through.

Advantageously, the device 100 comprises also a one-way valve 40 which is located at the first end 10-1 of the tubular member 10, which preferably extends outside the casing 30; in particular, the one-way valve 40 is located upstream the inlet port 31 of the casing 30. The one-way valve 40 may allow the air flow (and eventually also liquid flow) from a surrounding ambient into the tubular member 10 up to the envelope 20 (in particular during an inspiration phase), but not in the opposite direction, i.e. from the tubular member 10 to the surrounding ambient (in particular during an exhalation phase). In other words, the virucidal liquid is advantageously permanently resident in the envelope 20 and possibly temporarily also in the tubular member 10; in particular, the tubular member 10 will empty and fill with part of the virucidal liquid cyclically as the wearer inspires and exhales.

Advantageously, the device 100 comprises also a filter 50 which is located at the duct which passes through the outlet port 32, which preferably extends outside the casing 30; in particular, with reference to FIG. 6, the filter 50 is located downstream the outlet port 32 of the casing 30. Preferably, the filter 50 is a charcoal filter configured to capture any virucidal liquid aerosol that may get out of the envelope 20. It is to be noted that if the device 100 is integrated in a personal protective equipment (PPE), for example in a protective face mask, the filter 50 is fluidly coupled to a protected atmosphere defined by the protective face mask (i.e. the volume around the mouth and the nose of the wearer covered by the protective face mask), so that the clean air delivered downstream the device 100, in particular downstream the filter 50, may be inhaled by the wearer. Advantageously, the air exhaled by the wearer subsequently exits to the ambient atmosphere separately, in particular through a suitable one-way valve, which is typically provided at the protective face mask, to let the air out from the protective face mask. Another one-way valve may be provided at the protective face mask to avoid that any air may flow back from the volume around the mouth and the nose of the wearer into the device 100 first through the filter 50 (if any) and then through the outlet port 32.

It is also to be noted that if the device 100 is integrated in a personal protective equipment (PPE), for example in a protective face mask, the device 100 requires no power source: the air may flow through the device 100 thanks to a wearer which draws in air as he/she inhales.

1. A device for removing virus particles in an air flow, wherein the device comprises:
a tubular member having an inlet at a first end portion and a plurality of outlets at an outer lateral surface;
an envelope surrounding totally or partially the tubular member and containing virucidal liquid, wherein the envelope comprises a membrane being gas-permeable and liquid-impermeable;
the device being configured so that air enters through the inlet, flows in the tubular member, escapes through the outlets, diffuses in the virucidal liquid, and exits through the gas-permeable and liquid-impermeable membrane of the envelope, the tubular member being configured so that virucidal liquid cannot escape from the inlet (11).

2. The device of claim 1, comprising further:
a casing having an inlet port for receiving the air flow before virus removal and an outlet port for sending the air flow after virus removal, wherein the casing houses the tubular member and the envelope;
wherein the inlet port is fluidly coupled to the inlet, and
wherein the outlet port is fluidly coupled to the gas-permeable and liquid-impermeable membrane of the envelope.

3. The device of claim 1, wherein the virucidal liquid is a surfactant solution, in particular a benzalkonium chloride solution.

4. The device of claim 1, wherein the tubular member (10) comprises further a plurality of ribs configured to support the envelope.

5. The device of claim 4, wherein one or more or all of the ribs of said plurality is in the form of a disk or a ring.

6. The device of claim 4, wherein the plurality of ribs are mechanically coupled to an outer lateral surface of the tubular member.

7. The device of claim 4, wherein the ribs have an increasing size depending on a distance from the inlet.

8. The device of claim 1, wherein the outlets are holes having an increasing size depending on a distance from the inlet.

9. The device of claim 1, wherein the gas-permeable and liquid-impermeable membrane is positioned so to face the outlets.

10. The device of claim 1, wherein the envelope is in the form of a sack having a mouth sealed to the first end portion (10-1) of the tubular member.

11. The device of claim 1, wherein the tubular member is rolled up so to form in particular a solenoid or a spiral.

12. The device of claim 1, wherein the envelope is rolled up.

13. The device of claim 1, comprising further a one-way valve fluidly coupled upstream to inlet port.

14. The device (100) of claim 1, comprising further a filter fluidly coupled downstream to the outlet port.

15. A personal protective equipment comprising a device having the features set out in claim 1.
